(19) United States
(12) Patent Application Publication
(10) Pub. No.: US 2019/0004048 A1
(43) Pub. Date: Jan. 3, 2019

(54) Biomarker of Survival in the Treatment of Renal Cell Carcinoma with a VEGFR Inhibitor and an Ang2 Inhibitor

(71) Applicant: AMGEN INC., Thousand Oaks, CA (US)

(72) Inventors: Abraham Antonio ANDERSON, Sherman Oaks, CA (US); Bruce A. BACH, Thousand Oaks, CA (US); Cheryl A. PICKETT-GIES, Billings, MT (US); Michael B. BASS, Thousand Oaks, CA (US)

(73) Assignee: AMGEN INC., Thousand Oaks, CA (US)

(21) Appl. No.: 15/737,850

(22) PCT Filed: Jun. 22, 2016
(86) PCT No.: PCT/US2016/038778

§ 371 Date: Dec. 19, 2017

Related U.S. Application Data
(60) Provisional application No. 62/185,482, filed on Jun. 26, 2015.

(51) Int. Cl. G01N 33/574; A61K 38/04; A61P 35/00; G01N 33/50

(52) U.S. Cl. CPC G01N 33/57438; A61K 38/04; G01N 2800/52; G01N 33/5008; A61P 35/00

(57) ABSTRACT

The present invention relates to methods, compositions, and kits for using placental growth factor (PLGF) as an informative biomarker in determining the clinical benefit to renal cell carcinoma patients by treatment with a VEGFR inhibitor and an Ang2 inhibitor.

### PRIORITY

This application claims benefit to U.S. Provisional Application No. 62/185,482, tiled Jun. 26,2015, the contents of which are hereby incorporated by reference in its entirety.

### FIELD OF THE INVENTION

The present invention relates to methods, compositions, and kits for using placental growth factor (PLGF) as an informative biomarker in determining the clinical benefit of treatment with a VEGFR inhibitor and an Ang2 inhibitor to renal cell carcinoma patients.

### BACKGROUND OF THE INVENTION

The American Cancer Society estimates that in 2015 approximately 61,560 new cases of kidney cancer will occur and that approximately 14,080 people will die from the disease. See, “Kidney Cancer (Adult)—Renal Cell Carcinoma,” American Cancer Society (2015) (www.cancer.org/acs/groups/cid/documents/webcontent/003107-pdf.pdf). Renal cell carcinoma (RCC) accounts for 2% to 3% of all malignant diseases in adults, is the most common form of kidney cancer, and is responsible for approximately 90-95% of kidney cancer cases. It is the seventh most common cancer in men and the ninth most common cancer in women. Sew Siegel et al, “Cancer statistics,” CA Cancer J Clin., 62(1): 10-29 (2012). Although detection of kidney tumors has improved, the rate of RCC-related mortality has increased. See, Rint et al., “Renal cell carcinoma,” Lancet., 373(9669): 1119-1132 (2009); and Hollingsworfh et al., “Rising incidence of small renal masses: a need to reassess treatment effect,” J. Natl. Cancer Inst., 98(18): 1331-1334 (2006).

Angiogenesis plays a crucial, role in RCC tumor progression. Nascent and small tumors can obtain sufficient oxygen and nutrients to sustain their growth by simple diffusion. Beyond, a diameter of 1 to 2 mm, however, diffusion cannot provide these elements in the amounts required for further growth. For growth beyond that size, tumor growth requires angiogenesis. Angiogenesis, accordingly, has been seen as a promising target for developing an effective general treatment for tumors.

Three principal mechanisms play an important part in the activity of angiogenesis inhibitors against tumors: (i) inhibition of the growth of vessels, especially capillaries, into avascular resting tumors, with the result that there is no net tumor growth owing to the balance that is achieved between cell death and proliferation; (ii) prevention of the migration of tumor cells owing to the absence of blood flow to and from tumors; and (iii) inhibition of endothelial cell proliferation, thus avoiding the paracrine growth-stimulating effect exerted on the surrounding tissue by the endothelial cells which normally line the vessels. See. Connell et al., Exp. Opin. Ther. Patents, 11:77-114 (2001).

One of the best-characterized systems implicated in the regulation of angiogenesis—the endothelial cell-selective signal, transduction system involves the Tie2 receptor tyrosine kinase (NCBI Reference No. NP_00450.2; referred to as “Tie2” or “Tie2R” (also referred to as “ORK”); murine Tie2 is also referred to as “tek”) and its ligands, the angiopoietins (Gale, N. W. and Yancopoulos, G. D., Genes Dev. 13:1055-1066 [1999]). Indeed, most endothelial cell-selective signal transduction systems involve the Tie2 receptor tyrosine kinase and the angiopoietins. There are 4 known angiopoietins; angiopoietin-1 (“Ang1”) through angiopoietin-4 (“Ang4”), These angiopoietins are also referred to as “Tie2 ligands.”

Vascular endothelial growth factor (VEGF) is also associated with tumor angiogenesis. VEGF is a dimeric, disulfide-linked 46-kDa glycoprotein related to “Platelet-Derived Growth Factor” (PDGF) and produced by normal cell lines and tumor cell lines. Other members of the VEGF family include “Placental Growth factor” (PLGF) and VEGF-C. VEGF receptors (VEGFR) are transmembranous receptor tyrosine kinases characterized by an extracellular domain with seven immunoglobulin-like domains and an intracellular tyrosine kinase domain. Various types of VEGF receptor are known including VEGFR-1 (also known as flt-1), VEGFR-2 (also known as KDR), and VEGFR-3.

VEGF is an endothelial cell-specific mitogen that exhibits angiogenic activity in vivo; is chemotactic for endothelial cells and monocytes; and induces plasminogen activators in endothelial cells, which are involved in the proteolytic degradation of extracellular matrix during the formation of capillaries. A number of isoforms of VEGF are known, which show comparable biological activity, but differ in the type of cells that secrete them and in their heparin-binding capacity.

There currently exists a need for improved methods of treating patients with RCC. In addition, there exists a need for determining whether particular patients are likely to respond to a treatment regimen. An understanding of whether a patient is likely to respond to a treatment regimen reduces the risk of exposing patients to treatments which are unlikely to provide a treatment benefit and aids in the allocation of healthcare resources in a manner most beneficial to patients. To this end, an understanding of biomarkers informative of whether a patient is likely to benefit from a particular RCC treatment regimen will aid in the provision of the appropriate treatment to such patients. In one aspect, the present invention addresses this need by, for example, aiding in the treatment of human RCC patients with a combination of a VEGFR inhibitor and an Ang2 Inhibitor.

### SUMMARY OF THE INVENTION

In one aspect the present invention provides a method of determining whether human renal cell carcinoma (RCC) patients having an increased likelihood of obtaining clinical benefit from, treatment with a therapeutically effective amount of a VEGFR inhibitor and an Ang2 inhibitor. The method comprises measuring the concentration of PLGF in an RCC patient sample (e.g., serum or plasma), and determining that the PLGF concentration in the patient sample (i.e., the patient PLGF concentration) is lower than a PLGF-reference concentration, wherein a patient with a patient PLGF concentration lower than the PLGF reference concentration has an increased likelihood of obtaining clinical benefit from treatment with a therapeutically effective amount of a VEGFR inhibitor and an Ang2 inhibitor. In some embodiments, the method includes obtaining an RCC sample from the patient.

Another aspect of the present invention provides a method of treating human RCC patients with a therapeutically effective amount of a VEGFR inhibitor and an Ang2 inhibitor. The method comprises measuring the concentration of PLGF in an RCC patient sample (e.g., serum or plasma), determining that the PLGF concentration in the RCC patient sample (i.e., the patient PLGF concentration) is lower than a PLGF reference concentration, and administering a therapeutically effective amount of a VEGFR inhibitor and an Ang2 inhibitor to the patient. In some embodiments, the method includes obtaining an RCC sample from the patient.

In another aspect, the present invention provides a method of treating human RCC patients having a patient PLGF concentration lower than a PLGF reference concentration, the method comprising administering a therapeutically effective amount of a VEGFR inhibitor and an Ang2 inhibitor to the patient. In some embodiments, it has already been determined that the human RCC patients have a patient PLGF concentration lower than a PLGF reference concentration. In some embodiments, the patient PLGF concentration and PLGF reference concentration are serum concentrations. In a particular embodiment, the VEGFR inhibitor is sunitinib and the Ang2 inhibitor is trebananib.

The present invention also provides a method of treating human RCC patients having an increased likelihood of obtaining clinical benefit from treatment with a therapeutically effective amount of a VEGFR inhibitor and an Ang2 inhibitor. The method comprises measuring the concentration of PLGF in an RCC patient sample (e.g., serum or plasma), determining that the PLGF concentration in the patient sample (i.e., the patient PLGF concentration) is lower than a PLGF reference concentration, and administering a therapeutically effective amount of a VEGFR inhibitor and an Ang2 inhibitor to the patient. In some embodiments, the method includes obtaining an RCC sample from the patient.

In another aspect, the present invention provides a method of identifying a human RCC patient having an increased likelihood of obtaining clinical benefit from treatment with a therapeutically effective amount of a VEGFR inhibitor and an Ang2 inhibitor. The method comprises measuring the concentration of PLGF in an RCC patient sample (e.g., serum or plasma) and determining that the PLGF concentration in the patient sample (i.e., the patient PLGF concentration) is lower than a PLGF reference concentration, wherein the patient with a patient PLGF concentration lower than the PLGF reference concentration has an increased likelihood of obtaining clinical benefit from treatment with a therapeutically effective amount of a VEGFR inhibitor and an Ang2 inhibitor. In some embodiments, the method includes obtaining an RCC sample from the patient.

The PLGF reference concentration can be the median or mean serum PLGF concentration as determined from RCC patient samples. The patient PLGF concentration can be determined from serum, plasma, or urine. As a general principle, the patient PLGF concentration and the PLGF reference concentration should be determined from the same type of sample. That is, if the patient PLGF concentration is determined from serum, the PLGF reference concentration should also be determined from serum.

In some embodiments, the VEGFR inhibitor is bevacizumab, pazopanib, sunitinib, axitinib, ponatinib, cabozantinib, lenvatinib, ramucirumab, regorafenib, vandetanib, or ziv-aflibercept. In a specific embodiment, the VEGFR inhibitor is sunitinib.

The Ang2 inhibitor can be a dual Ang2 and Ang1 inhibitor. In some embodiments the Ang2 inhibitor is a binding polypeptide which can be, for example, an anti-Ang2 antibody, a soluble Tie2-Fc fusion polypeptide, or an anti-Tie2 antibody. In some embodiments, a bispecific binding polypeptide is an anit-VEGFR and anti-Ang2 binding polypeptide. In some embodiments, the ANG2 inhibitor is CVX-060, MEDI3617, DX-2240, REGN910, AZD-5180, CGI-1842, LC06, CGEN-25017, RG7594, CVX-241, TAvi6m, H4L4, or trebananib (also referred to as AMG 386 or 2XCon4C). In another embodiment, the ANG2 inhibitor is H4L4, or trebananib.

In a particular embodiment, the VEGFR inhibitor is sunitinib and the ANG2 inhibitor is trebananib.

### BRIEF DESCRIPTION OF THE DRAWINGS

FIGS. 1-4 are Kaplan-Meier plots showing progression free (PFS) and overall survival (OS) differences between subjects with patient serum PLGF concentrations above and below a serum PLGF reference concentration. Baseline serum PLGF was measured in 83 patients. Of those patients, 42 were administered oral sunitinib 50 mg once daily (QD) 4-weeks-on/2-weeks-off and intravenous trebananib QW at 10 mg/kg; while 41 were administered oral sunitinib 50 mg once daily (QD) 4-weeks-on/2-weeks-off and intravenous trebananib QW at 15 mg/kg. Dosing regimens and protocols are further described in the Examples.

FIG. 1 shows data from all 83 patients demonstrating increased PFS in patients with baseline patient serum PLGF concentrations <31.20 pg/mL, compared to patients with baseline patient serum PLGF concentrations >31.20 pg/mL. The data yielded a Cox proportional hazard ratio (HR) of 2.23 (CI 1.08-3.09), p=0.026, and a 7.3 month difference in median PFS. 31.20 pg/mL represents the median patient serum PLGF concentration.

FIG. 2 shows data from all 83 patients demonstrating increased OS in patients with baseline patient serum PLGF concentrations <31.20 pg/mL, compared to patients with baseline patient serum PLGF concentrations >31.20 pg/mL. The data yielded a Cox proportional hazard ratio (HR) of 2.5. (CI 1.23-5.25), p=0.012, and approximately a 21 month difference in median OS. 31.20 pg/mL represents the median patient serum PLGF concentration.

FIG. 3 shows data from all 83 patients demonstrating increased PFS in patients with baseline patient serum PLGF concentrations <28.90 pg/mL, compared to patients with baseline patient serum PLGF concentrations >28.90 pg/mL. The data yielded a Cox proportional hazard ration (HR) of 3.00 (CI 1.34-4.2), p=0.003, and an 8.8 month difference in median PFS. 28.90 pg/mL represents the optimal patient serum PLGF concentration according to the dataset.

FIG. 4 shows data from all 83 patients demonstrating increased OS in patients with baseline patient serum PLGF concentrations <28.90 pg/mL, compared to patients with baseline patient serum PLGF concentrations >28.90 pg/mL. The data yielded a Cox proportional hazard ration (HR) of 2.82 (CI 1.42-7.01), p=0.005, and approximately a 28 month difference in median OS. 28.90 pg/mL represents the optimal patient serum PLGF concentration according to the dataset.

### DETAILED DESCRIPTION

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

The term “Ang1” or “human Ang1” refers to the polypeptide human angiopoietin 1, a ligand of the human Tie2 receptor. An “Ang1 inhibitor” refers to an Ang1-specific binding agent that specifically binds to human Ang1 and/or human Tie2 thereby inhibiting specific binding of Ang1 to the human Tie2 receptor.

The term “Ang2” or “human Ang2” refers to the polypeptide also called angiopoietin 2 set forth, for example, in FIG. 6 (SEQ ID NO: 6) of U.S. Pat. No. 6,166,185 (hereby incorporated by reference) (“Tie2 ligand-2”) (see also, National Center for Biotechnology Information (NCBI) Accession No. AAI126203) as well as related native (i.e., wild-type) polypeptides such as allelic variants or mature forms of the polypeptide (absent the signal peptide), or splice variants (isoforms).

The term “Ang2 inhibitor” refers to an Ang2-specific binding agent that binds to Ang2 and inhibits Ang2 binding to the Tie2 receptor. In some embodiments, the Ang2-specific binding agent binds to human Ang2, inhibits its binding to the human Tie2 receptor, and results in a statistically significant decrease in angiogenesis, as measured by at least one functional assay of angiogenesis. Examples of such functional assays of angiogenesis include but are not limited to, tumor endothelial cell proliferation or the corneal micropocket assays (see, Oliner et al. Cancer Cell 6:507-516, 2004). See also, U.S. Pat. Nos. 5,712,291 and 5,871,723 (all of which are incorporated by reference). As those of ordinary skill in the art are aware, a corneal micropocket assay can be used to quantify the inhibition of angiogenesis. In this assay, agents to be tested for angiogenic activity are absorbed into a nylon membrane, which is implanted into micropockets created in the corneal epithelium of anesthetized mice or rats. Vascularization is measured as the number and extent of vessel ingrowth from the vascularized corneal limbus into the normally avascular cornea. See, U.S. Pat. No. 6,248,327 which describes planar migration and corneal pocket assays (hereby incorporated by reference). In certain embodiments, the Ang2 inhibitor is an antibody, avimer (Nature Biotechnology 23, 1556-1561 (2005); hereby incorporated by reference), peptibody (Fc-peptide fusion protein), Fe-soluble Tie2 receptor fusion (i.e., a “Tie2 trap”), or small molecule Ang2 inhibitor.

The term “Ang2-specific binding agent” refers to a molecule that specifically binds to human Ang2 and inhibits Ang2 binding with Tie2. In some embodiments, this inhibition results in a statistically significant decrease in angiogenesis.

The term “antibody” refers to isolated forms of both glycosylated and non-glycosylated immunoglobulins of any isotype or subclass, including: 1) human (e.g., CDR-grafted), humanized, and chimeric antibodies; and 2) monospecific or multi-specific antibodies, monoclonal, polyclonal, irrespective of whether such antibodies are produced, in whole or in part, via immunization, through recombinant technology, by way of in vitro synthetic means, or otherwise. Thus, the term “antibody” is inclusive of those that are prepared, expressed, created or isolated by recombinant means, such as (a) antibodies isolated from an animal (e.g., a mouse) that is transgenic for human immunoglobulin genes or a hybridoma prepared therefrom; (b) antibodies isolated from a host cell transfected to express the antibody (e.g., from a transfectoma); (c) antibodies isolated from a recombinant, combinatorial antibody library; and (d) antibodies prepared, expressed, created or isolated by any other means that involve splicing of immunoglobulin gene sequences to other DNA sequences. Antibodies may be monoclonal antibodies, such as humanized or fully-human monoclonal antibodies. Typically, antibodies will be IgG1 or IgG2 subclass antibodies. The antibody may bind human Ang2 or human Tie2 with a Kd of less than about 10 nM, 5 nM, 1 nM, or 500 pM.

The term “bispecific molecule” refers to any agent, e.g., a protein, peptide, or protein or peptide complex, which is able to bind at least two different targets. For example, the bispecific molecule may specifically bind to two distinct epitopes of the same protein or two epitopes located on two distinct proteins.

The term “multispecific molecule” or “heterospecific molecule” refers to any agent, e.g., a protein, peptide, or protein or peptide complex, which has two or more different binding specificities. Accordingly, the invention includes, but is not limited to, bispecific, trispecific, tetraspecific, and other multispecific molecules.

The term “binding polypeptide” refers to a molecule that comprises a polypeptide wherein the polypeptide specifically binds to a target. Exemplary binding polypeptides include: antibodies, peptibodies, avimers, Fc-soluble receptor fusion ligand trap (e.g., an Fc-soluble Tie2 fusion), CovX-bodies (see, WO 2008/056346), or specifically binding peptides (such as those obtained from screening a peptide library). A binding polypeptide of the present invention includes those that bind to a single epitope as well as multispecific binding polypeptides that bind to two epitopes (bispecific), three (trispecific), four (tetraspecific), or more epitopes.

As used herein, the term “clinical benefit” in the context of treating human RCC refers to a statistically significant decrease in at least one of: the rate of tumor growth, a cessation of tumor growth, or in a reduction in the size, mass, metabolic activity, or volume of the tumor, as measured by standard criteria such as, but not limited to, the Response Evaluation Criteria for Solid Tumors (RECIST), or a statistically significant increase in survival (PFS and/or OS) relative to treatment with a control.

The terms “effective amount” and “therapeutically effective amount,” in the context of the present invention, refer to an amount of a compound or combination of compounds which: (a) inhibits cancer (e.g., RCC) progression in a population of cancer patients (e.g., RCC patients); and/or (b) increases the length of time for progression-free survival (PFS), overall survival (OS), or both of a patient with cancer (e.g., RCC). Those of skill will recognize that the effective amount or therapeutically effective amount is determined from a patient population and therefore, although an individual patient may or may not obtain clinical benefit from a therapeutically effective amount, a statistically significant number of patients in the relevant patient population will obtain clinical benefit. In one example, the terms “effective amount” and “therapeutically effective amount” refer to an amount of a combination of an Ang2 inhibitor and a VEGFR inhibitor which; (a) inhibits cancer (e.g., RCC) progression in a population of cancer patients (e.g., RCC patients); and/or (b) increases the length of time for progression-free survival (PFS), overall survival (OS), or both of a patient with cancer (e.g., RCC).

The term “Fc” in the context of an antibody or peptibody is typically a fully human Fc, and may be any of the immunoglobulins (e.g., IgG1 and IgG2). Fc molecules that are partially human or obtained from non-human species are also included herein.

The term “Fc-peptide fusion” refers to a peptide that is covalently bonded, directly or indirectly, to an Fc. Exemplary Fc-peptide fusion molecules include a peptibody such as those disclosed in WO 03/057134 (hereby incorporated by reference) as well as an Fc covalently bonded, directly or indirectly, to an Ang2 specific binding fragment of the Tie2 receptor.

The term “human antibody” refers to an antibody in which both the constant and framework regions consist of fully or substantially all human sequences.

The term “humanized antibody” refers to an antibody in which all or substantially all of the constant region is derived from or corresponds to human immunoglobulins, while all or part of one or more variable regions is derived from another species, for example a mouse.

The term “increased likelihood” or “increased likelihood of obtaining clinical benefit” means a statistically significant probability of obtaining clinical benefit by a group of treated individuals after a specified treatment relative to a control group. Exemplary statistical tests include, but are not limited to, the Cox proportional hazards test of PFS or OS (yielding a p-value of equal to or less than 0.05).

The term “monoclonal antibody” or “monoclonal antibody composition” as used herein refers to a preparation of antibody molecules of single molecular composition. A monoclonal antibody composition displays a single binding specificity and affinity for a particular epitope. The term “human monoclonal antibody” refers to antibodies displacing a single binding specific which have variable and constant regions derived from human germline immunoglobulin sequences. The term “monoclonal” is not limited to any particular method for making an antibody.

The term “overall survival” (OS) refers to the fraction of subjects in an arm of a clinical trial who are alive at a given point in time following treatment with an active agent for the disease (e.g., renal cell carcinoma).

The terms “peptide,” “polypeptide,” or “protein” are used interchangeably throughout and refer to a molecule comprising two or more amino acid residues joined to each other by peptide bonds. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “peptibody” refers to a specific binding agent that is a molecule comprising an antibody Fc domain attached to at least one peptide. The production of peptibodies is generally described in PCT publication WO 00/24782 (published May 4, 2000 and incorporated herein by reference). Exemplary peptides may be generated by any of the methods set forth therein, such as carried in a peptide library (e.g., a phage display library), generated by chemical synthesis, derived by digestion of proteins, or generated using recombinant DNA techniques.

The term “PLGF” or “Placental Growth Factor” refers to human placental growth factor, a member of the VEGF family of growth factors and a specific ligand of VEGFR-1. PLGF in relation to this invention is meant to include in the four known isoforms, PLGF-1, PLGF-2, PLGF-3 and PLGF-4. See, NCBI Accession No. NP 002623.

The term “PLGF reference concentration” refers to a PLGF concentration to which a patient PLGF concentration is compared.

The term “patient PLGF concentration” refers to the concentration of PLGF in cancer patient (e.g., an RCC patient). The concentration can be measured in a sample obtained from a patient such as, e.g., tissue or fluids (including, but not limited to, plasma, serum, or urine).

The term “predictive” or “predicting” in the context of a biomarker, such as PLGF, means that the biomarker provides a means of identifying, directly or indirectly, an increased likelihood of a patient obtaining clinical benefit (e.g., PFS and/or OS) upon therapeutic treatment, such as treatment with a therapeutically effective amount of a VEGFR inhibitor and an Ang2 inhibitor. Thus, in this context the present invention provides a means of “identifying” or “determining” an RCC patient having an increased likelihood of clinical benefit prior to being administered a therapeutically effective amount of a VEGFR inhibitor and an Ang2 inhibitor of the invention. Conversely, the term can also be applied to situations in which the biomarker provides a means of predicting, directly or indirectly, patients who are statistically likely to obtain less clinical benefit from such treatment relative to a control.

The term “progression free survival” (PFS) refers to the duration of time from the start of treatment to the time of progression of disease (measured radiographically or clinically) or death, whichever occurs first.

The term “prognostic” in the context of a biomarker means that the biomarker identifies an increased likelihood of a patient obtaining clinical benefit regardless of treatment.

The term “renal cell carcinoma” or “RCC” or “advanced renal cell carcinoma” or “advanced RCC” refers to human kidney cancer typically classified as being of at least one of the following histologies: clear cell carcinoma, papillary renal carcinoma (type 1 or type 2), chromophobe renal carcinoma, oncocytoma.

The term “specifically binds” refers to the ability of, e.g., a specific binding agent of the present invention, under specific binding conditions, to bind a target molecule such that its affinity is at least 10 times as great as the average affinity of the same specific binding agent to a collection of random peptides or polypeptides. In some embodiments, the specific binding agent binds a target molecule such that its affinity is 50, 100, 250, 500, or 1000 times as great as the average affinity of the same specific binding agent to a collection of random peptides or polypeptides. A specific binding agent need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a molecule having a substantially similar epitope as the target molecule (e.g., a paralog) is within the scope of the term “specific binding” which is determined relative to a statistically valid sampling of unique non-targets (e.g., random polypeptides). Thus, a specific binding agent of the invention may specifically bind to more than one distinct species of target molecule, such as specifically binding to both Ang2 and Ang1. Solid-phase ELISA immunoassays can be used to determine specific binding. Generally, specific binding proceeds with an association constant of at least about 1×10⁷ M⁻¹, and often at least 1×10⁸ M⁻¹, 1×20⁹ M⁻¹, or, 1×10¹⁰ M⁻¹.

The term “Tie2-specific binding agent” refers to a molecule that specifically binds to human Tie2 and inhibits its binding with Ang2 and/or inhibits human Tie2 signal transduction resulting in a statistically significant decrease in angiogenesis, as measured by at least one functional assay of angiogenesis such as tumor endothelial cell proliferation or the corneal micropocket assay (Oliner et al. Cancer Cell 6:507-516, 2004; and U.S. Pat. Nos. 5,712,291 and 5,871,723; all of which are incorporated herein by reference). In certain embodiments, the Tie2 inhibitor is an antibody, avimer (Nature Biotechnology 23, 1556-1561 (2005) (incorporated herein by reference)), peptibody, or small molecule Ang2 inhibitor.

The term “VEGFR” refers to human vascular endothelial factor receptors (VEGFR) including VEGFR-1, VEGFR-2, and VEGFR-3.

The term “VEGFR inhibitor” refers to a molecule that inhibits the interaction between VEGF, the native, endogenous ligand of human vascular endothelial growth factor receptor (VEGFR), with a VEGFR. Generally, a VEGFR inhibitor will interfere with signaling between at least one VEGFR and at least one native ligand VEGF (vascular endothelial growth factor) so as to inhibit angiogenesis. A VEGFR inhibitor may be a VEGF tyrosine kinase angiogenesis inhibitor. The VEGFR inhibitors of the present invention do not include sorafenib.

### Methods of the Present Invention

In one aspect the present invention provides a method of determining whether human renal cell carcinoma (RCC) patients having an increased likelihood of obtaining clinical benefit from treatment with a therapeutically effective amount of a VEGFR inhibitor and an Ang2 inhibitor. The method comprises measuring the concentration of PLGF in an RCC patient sample (e.g., serum or plasma), and determining that the PLGF concentration in the patient sample (i.e., the patient PLGF concentration) is lower than a PLGF reference concentration, wherein a patient with a patient PLGF concentration lower than the PLGF reference concentration has an increased likelihood of obtaining clinical benefit from treatment with a therapeutically effective amount of a VEGFR inhibitor and an Ang2 inhibitor. In one embodiment, both the patient PLGF concentration and the PLGF reference concentration are measured from the serum. In a particular embodiment, the VEGFR inhibitor is sunitinib and the Ang1 inhibitor is trebananib. In some embodiments, the method includes obtaining an RCC sample from the patient.

Another aspect of the present invention provides a method of treating human RCC patients with a therapeutically effective amount of a VEGFR inhibitor and an Ang2 inhibitor. The method comprises measuring the concentration of PLGF in an RCC patient sample (e.g., serum or plasma), determining that the PLGF concentration in the RCC patient sample (i.e., the patient PLGF concentration) is lower than a PLGF reference concentration, and administering a therapeutically effective amount of a VEGFR inhibitor and an Ang2 inhibitor to the patient. In one embodiment, both the patient PLGF concentration and the PLGF reference concentration are measured from the serum. In a particular embodiment, the VEGFR inhibitor is sunitinib and the Ang2 inhibitor is trebananib. In some embodiments, the method includes obtaining an RCC sample from the patient.

In another aspect, the present invention provides a method of treating human RCC patients having a patient PLGF concentration lower than a PLGF reference concentration, the method comprising administering a therapeutically effective amount of a VEGFR inhibitor and an Ang2 inhibitor to the patient. In some embodiments, it has already been determined that the human RCC patients have a patient PLGF concentration lower than a PLGF reference concentration. In some embodiments, the patient PLGF concentration and PLGF reference concentration are serum concentrations. In a particular embodiment the VEGFR inhibitor is sunitinib and the Ang2 inhibitor is trebananib.

The present invention also provides a method of treating human RCC patients having an increased likelihood of obtaining clinical benefit from treatment with a therapeutically effective amount of a VEGFR inhibitor and an Ang2 inhibitor. The method comprises measuring the concentration of PLGF in an RCC patient sample (e.g., serum or plasma), determining that-the PLGF concentration in the patient sample (i.e., the patient PLGF concentration) is lower than a PLGF reference concentration, and administering a therapeutically effective amount of a VEGFR inhibitor and an Ang2 inhibitor to the patient. In one embodiment, both the patient PLGF concentration and the PLGF reference concentration are measured from the serum. In a particular embodiment, the VEGFR inhibitor is sunitinib and the Ang2 inhibitor is trebananib. In some embodiments, the method includes obtaining an RCC sample from the patient.

In another aspect, the present invention provides a method of identifying a human RCC patient having an increased likelihood of obtaining clinical benefit from treatment with a therapeutically effective amount of a VEGFR inhibitor and an Ang2 inhibitor. The method comprises measuring the concentration of PLGF in an RCC patient sample (e.g., serum or plasma) and determining that the PLGF concentration in the patient sample (i.e., the patient PLGF concentration) is lower than a PLGF reference concentration, wherein the patient with a patient PLGF concentration lower than the PLGF reference concentration has an increased likelihood of obtaining clinical benefit from treatment with a therapeutically effective amount of a VEGFR inhibitor and an Ang2 inhibitor. In one embodiment, both the patient PLGF concentration and the PLGF reference concentration are measured from the serum. In a particular embodiment, the VEGFR inhibitor is sunitinib and the Ang2 inhibitor is trebananib. In some embodiments, the method includes obtaining an RCC sample from the patient.

Those of skill will recognize that RCC patients having a patient PLGF concentration lower than the PLGF reference concentration can be identified indirectly as well as directly. Thus, by identifying those RCC patients from a group of RCC patients who have a higher patient PLGF concentration than die PLGF reference concentration one implicitly also identifies those that have a patient PLGF concentration equal to or lower than the PLGF reference concentration. Likewise, one can identify RCC patients with a patient PLGF concentration higher than the PLGF reference concentration by identifying the RCC patients in a group of RCC patients by a similar implicit process. Thus, the method of the invention extends to identification of both groups, one directly and one indirectly or implicitly.

### PLGF Reference Concentration

The PLGF (placental growth factor) reference concentration provides a reference value to which a patient's PLGF concentration can be compared. It has been discovered that RCC patient(s) with a patient PLGF concentration lower than a PLGF reference concentration exhibit greater PFS and OS after treatment with a VEGFR inhibitor and an Ang2 inhibitor (e.g., sunitinib and trebananib), compared to RCC patient(s) with a patient PLGF concentration higher than a PLGF reference concentration.

The PLGF reference concentration is a PLGF concentration determined from a plurality of RCC patients. From the resulting distribution of PLGF concentration values a PLGF reference concentration is calculated. The RCC patients who are assessed to determine the PLGF concentration generally have their PLGF concentration determined prior to treatment with a combination of the VEGFR inhibitor and Ang2 inhibitor, or after sufficient time has transpired that the PLGF concentration values obtained from the RCC patients are not significantly affected by the combination treatment or other treatment (i.e., after sufficient washout). For example, the PLGF concentration can be measured in RCC patients and used to determine the PLGF reference concentration if at least 15, 20, 30, 40, 50, 60, or 75 days have transpired since having been administered an Ang2 inhibitor and/or a VEGFR inhibitor or after other treatment that has substantially affected the PLGF concentration.

The number of RCC patients employed in determining the PLGF reference concentration can vary but is generally a sufficient number to obtain a statistically meaningful value. In some embodiments, the PLGF reference concentration is a value obtained from a statistical sampling of at least 10, 20, 30, 40, 50, 75, 100, 200, 300, 500, or 1000 RCC patients. RCC patients may have a statistically proportional representation of RCC histologies but can also be chosen such that at least 75%, 80%, 85%, 90%, 95%, or 100% of the patients have clear cell carcinoma.

In some embodiments, the PLGF concentration is determined from whole blood of the RCC patients. In other embodiments the PLGF reference concentration is determined from components of whole blood (such as from serum or plasma), or from urine. Methods for determining PLGF concentration from whole blood, serum, plasma, or urine are known in the art. Whole blood, serum, plasma, and urine PLGF concentrations can be analyzed, for example, by sandwich enzyme-linked immunosorbent assay (ELISA) and by an electrochemiluminescent multiplexed sandwich immunoassay (Meso-Scale Discovery [MSD], Gaithersburg, Md.). See also, for example, Quantikine® human PLGF immunoassay which can be used to assay PLGF concentration in whole blood, serum, plasma, and/or urine. Those of skill in the art will recognize that the specific method of determining the PLGF reference concentration should provide a value of sufficient accuracy and precision to allow a statistically meaningful comparison to the patient PLGF concentration. Furthermore, the skilled practitioner will recognize that the PLGF concentrations obtained from different methods or from different tissue biological samples (e.g., plasma and serum) can be used but the values will generally be normalized relative to each other so that the values can all be brought to a common scale.

The value of the PLGF reference concentration will may vary between patient populations selected for testing. Thus, for example, in some embodiments the value of the PLGF reference concentration when determined from human serum or plasma from a statistical, sampling of RCC patients (and as determined by electrochemiluminescent multiplexed sandwich immunoassay (Meso-Scale Discovery [MSD], Gathersburg Md.))

In some embodiments, the mean (average) value of PLGF concentration (serum or plasma) from the RCC patients is used for determining a PLGF reference concentration. In other embodiments, the PLGF reference concentration is within one standard deviation of the mean PLGF concentration of the RCC patients; often the value is the median PLGF concentration. As desired, more stringent values can be selected. Thus, in some embodiments in which clinical benefit is being determined the PLGF reference concentration is the value of the 25^{th}, 30^{th}, 35^{th}, 40^{th}, 45^{th}, 50^{th}, 55^{th}, 60^{th}, 65^{th}, 70^{th}, 75^{th}, 80^{th}, 85^{th} or 90^{th} percentile in the distribution. Thus, in some embodiments the clinician may desire to exclude the top 10^{th} percentile (i.e., the 90^{th} percentile) from treatment with the combination therapy of the present invention.

In other embodiments, the median value of PLGF concentration (serum or plasma) from the RCC patients is used for determining a PLGF reference concentration. In other embodiments, the PLGF reference concentration is within one standard deviation of the median PLGF concentration of the RCC patients. As desired, more stringent values can be selected. Thus, in some embodiments in which clinical benefit is being determined the PLGF reference concentration is the value of the 25^{th}, 30^{th}, 35^{th}, 40^{th}, 45^{th}, 50^{th}, 55^{th}, 60^{th}, 65^{th}, 70^{th}, 75^{th}, 80^{th}, 85^{th} or 90^{th} percentile in the distribution. Thus, in some embodiments the clinician may desire to exclude the top 10^{th} percentile (i.e., the 90^{th} percentile) from treatment with the combination therapy of the present invention.

Thus, in one embodiment, tire PLGF reference concentration is the mean PLGF serum or plasma concentration measured in a sample of RCC patients (e.g., the RCC patients in the clinical study described in the Examples). Thus, in some embodiments, the PLGF reference concentration is a PLGF serum concentration of about 20-35 pg/mL; about 25-35 pg/mL; about 27-33 pg/mL; about 27-32 pg/mL; about 28-32 pg/mL; about 29-32 pg/mL; about 30-32 pg/mL; or about 31-32 pg/mL. In some embodiments, the PLGF reference concentration is a PLGF serum concentration of 20-35 pg/mL; 25-35 pg/mL; 27-33 pg/mL; 27-32 pg/mL; 28-32 pg/mL; 29-32 pg/mL; 30-32 pg/mL; or 31-32 pg/mL. In other embodiments, the PLGF reference concentration is a PLGF serum concentration of about 25, about 26, about 27, about 28, about 29, about 30, about 31, about 32, about 33, about 34, or about 35 pg/mL. In other embodiments, the PLGF reference concentration is a PLGF serum concentration of 25, 26, 27, 28, 29, 30, 31, 32, 33, 34, or 35 pg/mL. In other embodiments, the PLGF reference concentration is a PLGF plasma concentration of about 20-35 pg/mL; about 25-35 pg/mL; about 27-33 pg/mL;. about 27-32 pg/mL; about 28-32 pg/mL; about 29-32 pg/mL; about 30-32 pg/mL; or about 31-32 pg/mL. In other embodiments, the PLGF reference concentration is a PLGF plasma concentration of 20-35 pg/mL; 25-35 pg/mL; 27-33 pg/mL; 27-32 pg/mL; 28-32 pg/mL; 29-32 pg/mL; 30-32 pg/mL; or 31-32 pg/mL. In yet other embodiments, the PLGF reference concentration is a PLGF plasma concentration of about 25, about 26, about 27, about 28, about 29, about 30, about 31, about 32, about 33, about 34, or about 35 pg/mL. In yet other embodiments, the PLGF reference concentration is a PLGF plasma concentration of 25, 26, 27, 28, 29, 30, 31, 32, 33, 34, or 35 pg/mL.

In a particular embodiment, the PLGF reference concentration is a PLGF serum concentration of about 31.20 pg/mL. In another embodiment, the PLGF reference concentration is a PLGF serum concentration of 31.20 pg/mL. In another particular embodiment, the PLGF reference concentration is a PLGF plasma concentration of about 31.20 pg/mL. In another embodiment, the PLGF reference concentration is a PLGF plasma concentration of 31.20 pg/mL.

The PLGF reference concentration may also be calculated to be the “optimal” PLGF serum or plasma concentration measured in the RCC patients in the clinical study described in the Examples. As discussed herein, the PLGF reference concentration represents the PLGF serum or plasma concentration below which RCC patients exhibit greater PFS and OS after treatment with a VEGFR inhibitor and an Ang2 inhibitor (e.g., sunitinib and trebananib), compared to RCC patient(s) with a patient PLGF concentration higher than the PLGF reference concentration. Thus, the PLGF reference concentration may be the “optimal” PLGF serum or plasma concentration—i.e., the PLGF concentration which, when used as a cutoff, yields the greatest difference in PES and/or OS between patients with PLGF concentrations higher and lower than the PLGF reference concentration. As would be appreciated by those in the field, the “optimal” PLGF serum or plasma concentration used in a clinical context may be adjusted by a clinician based on patient circumstances and clinical experience.

Thus, in some embodiments, the PLGF reference concentration is a PLGF serum concentration of about 20-35 pg/mL; about 25-35 pg/mL; about 25-33 pg/mL; about 26-32 pg/mL; about 27-31 pg/mL; about 28-30 pg/mL; or about 28-29 pg/mL. In some embodiments, the PLGF reference concentration is a PLGF serum concentration of 20-35 pg/mL; 25-35 pg/mL; 25-33 pg/mL; 26-32 pg/mL; 27-31 pg/mL; 28-30 pg/mL; or 28-29 pg/mL. In other embodiments, the PLGF reference concentration is a PLGF serum concentration of about 20, about 21, about 22, about 23, about 24, about 25, about 26, about 27, about 28, about 29, about 30, about 31, about 32, about 33, about 34, or about 35. In other embodiments, the PLGF reference concentration is a PLGF serum concentration of 20, 21, 22, 23, 24, 25, 26, 27, 28, 29, 30, 31, 32, 33, 34, or 35. In other embodiments, the PLGF reference concentration is a PLGF plasma concentration of about 20-35 pg/mL; about 25-35 pg/mL; about 25-33 pg/mL; about 26-32 pg/mL; about 27-31 pg/mL; about 28-30 pg/mL: or about 28-29 pg/mL. In other embodiments, the PLGF reference concentration is a PLGF plasma concentration of 20-35 pg/mL; 25-35 pg/mL; 25-33 pg/mL; 26-32 pg/mL; 27-31 pg/mL; 28-30 pg/mL; or 28-29 pg/mL. In yet other embodiments, the PLGF reference concentration is a PLGF plasma concentration of about 20, about 21, about 22, about 23, about 24, about 25, about 26, about 27, about 28, about 29, about 30, about 31, about 32, about 33, about 34, or about 35 pg/mL. In yet other embodiments, the PLGF reference concentration is a PLGF plasma concentration of 20, 21, 22, 23, 24, 25, 26, 27, 28, 29, 30, 31, 32, 33, 34, or 35 pg/mL.

In a particular embodiment, the PLGF reference concentration is a PLGF serum concentration of about 28.90 pg/mL. In another embodiment, the PLGF reference concentration is a PLGF serum concentration of 28.90 pg/mL. In another particular embodiment, the PLGF reference concentration is a PLGF plasma concentration of about 28.90 pg/mL. In another embodiment, the PLGF reference concentration is a PLGF plasma concentration of 28.90 pg/mL.

### Patient PLGF Concentration

The patient PLGF concentration is obtained from the RCC patient for whom treatment with a therapeutically effective dose of a VEGFR inhibitor and an Ang2 inhibitor is being considered. Typically, as is the case for the PLGF reference concentration, the measurement of the patient PLGF concentration is determined prior to treatment with a VEGFR inhibitor and/or an Ang2 inhibitor so as to obtain a value not altered by one or both agents. However, measurement of the patient PLGF concentration may occur subsequent to treatment with one or both agents if sufficient time has transpired to substantially reduce any affect of one or both of the agents, or of any other agent, on PLGF concentration levels. Thus, if treatment with one or both agents has taken place the patient PLGF concentration can be measured following cessation of treatment with the agent or agents significantly affecting the patients PLGF concentration. For example, the patient PLGF concentration can be measured after 1, 2, 3, 4, 5, 6, 7, 8 weeks cessation of treatment.

The patient PLGF concentration can be measured per the specific methods utilized for measuring the PLGF reference concentration. In some embodiments, the method utilized is the same for both the patient PLGF concentration and the PLGF reference concentration to ensure better correlation of measured values. Thus, for example, if the PLGF reference concentration is determined from whole blood, serum, plasma, or urine then the patient PLGF concentration will conveniently also be measured from whole blood, serum, plasma, or urine, respecitvely. Likewise, the specific assay method of measurement will generally also be substantially identical to minimize discrepancies. However, different biological samples and/or assay methods can also be utilized for determining the PLGF reference concentration and/or the patient PLGF concentration although values thereby obtained will typically be normalized relative to bring each value to a common scale.

Thus, in some embodiments, the patient PLGF concentration is measured from whole blood, serum, plasma, or urine. In other embodiments, the patient PLGF concentration is measured from serum or plasma. In a specific embodiment, the patient PLGF concentration is measured from serum. In another specific embodiment, the patient PLGF concentration is measured from plasma.

### Ang2 Inhibitors

The Ang2 inhibitors of the present invention, which are administered in combination with at least one VEGFR inhibitor of the invention, can be small molecules (less than about 1000 daltons) or large molecules (polypeptides of greater than about 1000 daltons). Exemplary Ang2 inhibitors include, but are not limited to, trebananib (i.e., AMG 386 or 2XCon4C) (Amgen Inc., see, e.g., U.S. Pat. No. 7,723,499), H4L4 (Amgen Inc.; see U.S. Ser. No. 12/378,993), CVX-060 (CovX/Pfizer), MEDI3617 (MedImmune/AstraZeneca), DX-2240 (Dyax/Sanofi-Aventis), REGN910 (Regeneron/Sanofi-Aventis), CGI-1842 (CGI Pharmaceuticals), LC06 (Roche), CGEN-25017 (Compugen), RG7594 (Roche), CVX-241 (CovX/Pfizer), LP-590 (Locus Pharmaceuticals), CEP-11981 (Cephalon/Sanofi-Aventis), MGCD265 (Methylgene), regorafenib (Bayer), or CrossMab (Roche). In a particular embodiment, the Ang2 inhibitor is trebananib.

In some embodiments, the Ang2 inhibitor is at least bispecific comprising an Ang2 inhibitor and a human DLL4 (delta like ligand 4) inhibitor (a “dual Ang2 and DLL4 inhibitor”). In some embodiments, the Ang2 inhibitor also inhibits Ang1 binding to the Tie2 receptor (a “dual Ang2 and Ang1 inhibitor”). The Ang2 inhibitors are inclusive of large molecules such as a peptide, peptibody, antibody, antibody binding fragment such as a F(ab) or F(ab′)2 fragment, an Fc-Tie2 extracellular domain (ECD) fusion protein (a “Tie2 trap”), and small molecules, or combinations thereof. In some embodiments, the dual Ang2 and Ang1 inhibitor is trebananib (Amgen Inc.) or H4L4 (Amgen Inc.). In some embodiments, the Ang2 inhibitor is at least bispecific, for example a dual Ang2 and DLL4 inhibitor. Methods for linking small or large molecule Ang2 inhibitors with other specific binding agents, such as a Ang2 inhibitor of the invention, are known in the art. Thus, for example, bispecific antibodies which act as dual Ang2 and Ang2 inhibitors of the invention can be made using known techniques.

In some embodiments, the Ang2 inhibitor is a binding polypeptide. Binding polypeptides may be produced by methods known to those of skill in the art such as by the modification of whole antibodies, or synthesized de novo using recombinant DNA technologies or peptide synthesis. Human or humanized antibodies or antigen binding regions can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Hanes and Plucthau PNAS USA 94:4937-4942 (1997) (ribosomal display), Parmley and Smith Gene 73:305-318 (1988) (phage display), Scott TIBS 17:241-245 (1992), Cwirla et al PNAS USA 87:6378-6382 (1990), Russel et al. Nucl. Acids Research 21:1081-1085 (1993), Hoganboom et al Immunol. Reviews 130:43-68 (1992), Chiswell and McCafferty TIBTECH 10:80-84 (1992), and U.S. Pat. No. 5,733,743 (all of which are hereby incorporated by reference).

### VEGFR Inhibitors

The VEGFR inhibitors of the present invention, which are administered in combination with at least one Ang2 inhibitor of the invention, can be small molecules (less than about 1000 daltons) or large molecules (polypeptides of greater than about 1000 daltons). Exemplary VEGFR inhibitors include, but are not limited to, bevacizumab, pazopanib, sunitinib, axitinib, ponatinib, caboxantinib, lenvatinib, ramucirumab, regorafenib, vandetanib, and ziv-aflibercept. In a specific embodiment, the VEGFR inhibitor is sunitinib. The VEGFR inhibitors of the present invention do not include sorafenib.

### Therapeutically Effective Dose of VEGFR Inhibitor and Ang2 Inhibitor

In the methods of the present invention, a therapeutically effective amount of the Ang2 inhibitor is administered in combination with a VEGFR inhibitor to RCC patients. The therapeutically effective dose of the specific binding agent can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The Ang2 inhibitor is administered at doses and rates readily determined by those of ordinary skill in the art. In some embodiments, the Ang2 inhibitor (e.g., trebananib) is administered to the patient at a dose ranging from about 0.3-30; about 1-25; about 1-20; about 5-20; about 1-15; about 5-15; or about 10-15 mg/kg of patient body weight. In some embodiments, the Ang2 inhibitor (e.g., trebananib) is administered to the patient at a dose ranging from 0.3-30; 1-25; 1-20; 5-20; 1-15; 5-15; or 10-15 mg/kg of patient body weight. In some embodiments, the Ang2 inhibitor (e.g., trebananib) is administered at doses of about 5, about 10, about 15, about 20, about 25, or about 30 mg/kg of patient body weight. In some embodiments, the Ang2 inhibitor (e.g., trebananib) is administered at doses of 5, 10, 15, 20, 25, or 30 mg/kg of patient body weight.

In particular embodiments, the Ang2 inhibitor (e.g., trebananib) is administered at doses of about 10 or about 115 mg/kg of patient body weight. In other particular embodiments, the Ang2 inhibitor (e.g., trebananib) is administered at doses of 10 or 15 mg/kg of patient body weight. In some embodiments, the Ang2 inhibitor (e.g., trebananib) is administered to the patient every 1, 2, 3, or 4 weeks. In a particular embodiment, the Ang2 inhibitor (e.g., trebananib) is administered every week.

In one embodiment, the Ang2 inhibitor (e.g., trebananib) is administered at 10 mg/kg of patient body weight, every week. In another embodiment, the Ang2 inhibitor (e.g., trebananib) is administered at 15 mg/kg of patient body weight, every week.

The VEGFR inhibitor is administered at doses and rates readily determined by those of ordinary skill in the art. In some embodiments, the VEGFR inhibitor (e.g., sunitinib) is administered to the patient at a dose ranging from about 1-100; about 1-90; about 1-80; about 1-75; about 10-75; about 20-75; about 25-75; about 35-65; about 40-60; or about 45-55 mg. In some embodiments, the VEGFR inhibitor (e.g., sunitinib) is administered to the patient at a dose ranging from 1-100; 1-90; 1-80; 1-75; 10-75; 20-75; 25-75; 35-65; 40-60; or 45-55 mg. In some embodiments, the VEGFR inhibitor (e.g., sunitinib) is administered to the patient at a dose of 1, 5, 10, 15, 20, 25, 30, 35, 40, 45, 50, 55, 60, 65, 70, 75, 80, 85, 90, 95, or 100 mg. In some embodiments, the VEGFR inhibitor (e.g., sunitinib) is administered to the patient at a dose of about 1, about 5, about 10, about 15, about 20, about 25, about 30, about 35, about 40, about 45, about 50, about 55, about 60, about 65, about 70, about 75, about 80, about 85, about 90, about 95, or about 100 mg. In other embodiments, the VEGFR inhibitor (e.g., sunitinib) is administered to tire patient at a dose of about 40, about 41, about 42, about 43, about 44, about 45, about 46, about 47, about 48, about 49, about 50, about 51, about 52, about 53, about 54, about 55, about 56, about 57, about 58, about 59, or about 60 mg. In other embodiments, the VEGFR inhibitor (e.g., sunitinib) is administered to the patient at a dose of 40, 41, 42, 43, 44, 45, 46, 47, 48, 49, 50, 51, 52, 53, 54, 55, 56, 57, 58, 59, or 60 mg.

Thus, in one aspect, the present invention relates to a therapeutically effective amount of an Ang2 inhibitor and a VEGFR inhibitor for use in treating human RCC patients having an increased likelihood of obtaining clinical benefit from treatment with a therapeutically effective amount of a VEGFR inhibitor and an Ang2 inhibitor. The invention also relates to a therapeutically effective amount of an Ang2 inhibitor and a VEGFR inhibitor for use in treating human RCC patients having a patient PLGF concentration lower than a PLGF reference concentration. In such embodiments, it may have been already determined that the RCC patient has a patient PLGF concentration lower than a PLGF reference concentration. In a particular embodiment, the VEGFR inhibitor is sunitinib and the Ang2 inhibitor is trebananib.

In a specific embodiment, the VEGFR inhibitor (e.g., sunitinib) is administered to the patient at a dose of about 50 mg. In another specific embodiment, the VEGFR inhibitor (e.g., sunitinib) is administered to the patient at a dose of 50 mg.

In one embodiment, the VEGFR inhibitor (e.g., sunitinib) is administered to the patient for a period of time, followed by a period of time without administration. For example, in one embodiment, the VEGFR inhibitor (e.g., sunitinib) is administered to the patient once daily for a period of 1, 2, 3, or 4 weeks, followed by a period of 1, 2, 3, or 4 weeks without administration. In a particular embodiment, the VEGFR inhibitor (e.g., sunitinib) is administered to the patient once daily for a period of 4 weeks, followed by a period of 2 weeks without administration (i.e., “4-weeks-on/2-weeks-off”),

The Ang2 inhibitor (e.g., trebananib) and VEGFR inhibitor (e.g., sunitinib) can be administered to a patient via any suitable route. Exemplary routes of administration include buccal, intra-arterial, intravenous, oral, parenteral, and subcutaneous administration. In specific embodiments, the Ang2 inhibitor (e.g., trebananib) is administered intravenously and the VEGFR inhibitor (e.g., sunitinib) is administered orally.

Specific treatment regimens useful in the methods of the present invention are illustrated the in the Examples. Such treatment regimens include, oral sunitinib 50 mg once daily (QD) 4-weeks-on/2-weeks-off and intravenous trebananib QW at 10 mg/kg; and oral sunitinib 50 mg once daily (QD) 4-weeks-on/2-weeks-off and intravenous trebananib QW at 15 mg/kg. Standard dosages and methods of administrations can be used, for example per the Food and Drug Administration (FDA) label.

The VEGFR Inhibitor of the present invention can be administered prior to and/or subsequent to (collectively, “sequential treatment”), and/or simultaneously with (“concurrent treatment”) the Ang2 inhibitor of the present invention. Sequential treatment (such as pretreatment, post-treatment, or overlapping treatment) of the combination, also includes regimens in which the drugs are alternated, or wherein, one component is administered long-term and the other(s) are administered intermittently. Components of the combination may be administered in the same or in separate compositions, and by the same or different routes of administration. Methods and dosing of administering chemotherapeutic agents are known in the art.

### Pharmaceutical Formulations and Kits

### Ang2 Inhibitor Formulations

A pharmaceutical composition comprising the Ang2 inhibitor (e.g., trebananib) of the present invention may suitable for intravenous administration and may contain formulation materials for modifying, maintaining or preserving, for example, the pH, osmolarity, viscosity, clarity, color, isotonicity, odor, sterility, stability, adsorption, or penetration of the composition.

The primary vehicle or carrier in a pharmaceutical composition may be either aqueous or non-aqueous in nature. For example, a suitable vehicle or carrier may be water for injection or physiological saline, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present invention, pharmaceutical compositions may be prepared for storage by mixing the selected composition having the desired degree of purity with optional formulation agents (Remington's Pharmaceutical Sciences, supra) in the form of a lyophilized cake or an aqueous solution. Further, the pharmaceutical composition may be formulated as a lyophilizate using appropriate excipients such as sucrose.

The formulation components are present in concentrations that are acceptable to the site of administration. For example, buffers are used to maintain the composition at physiological pH or at slightly lower pH, typically within a pH range of from about 5 to about 8. A particularly suitable vehicle for parenteral administration is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provide for the controlled or sustained release of the product which may then be delivered via a depot injection.

In another aspect, pharmaceutical formulations suitable for parenteral administration may be formulated in aqueous solutions (e.g., in physiologically compatible buffers such as Hanks' solution, ringer's solution, or physiologically buffered saline). Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration. In a specific embodiment, a lyophilized peptibody, such as trebananib, is formulated as disclosed in WO 2007/124090 (incorporated herein by reference).

### VEGFR Inhibitor Formulations

A pharmaceutical composition comprising the VEGFR inhibitor (e.g., sunitinib) may be suitable for oral administration. Suitable oral formulations typically comprise standard carriers (e.g., pharmaceutical grades of mannitol, lactose, starch, magnesium stearate, sodium saccharine, cellulose, and magnesium carbonate). Examples of suitable pharmaceutical carriers are described in Remington: the Science and Practice of Pharmacy, Alfonso R. Gennaro ed,. Mack Publishing Co. Easton, Pa., 19th ed., 1995, Chapters 87 and 88 (hereby incorporated by reference).

For example, oral VEGFR inhibitor (e.g., sunitinib) formulations may be prepared by combining the VEGFR inhibitor (e.g., sunitinib), oils, solvents, surfactants, and other components using well-known pharmaceutical formulation methods. The formulation of solid forms, such as powders, tablets, pills, and capsules is discussed in Remington: the Science and Practice of Pharmacy, Alfonso R. Gennaro ed., Mack Publishing Co. Easton, Pa., 19th ed., 1995, Chapters 91 and 92 (hereby incorporated by reference). The formulation of solutions, emulsions, and suspensions is discussed in Remington: the Science and Practice of Pharmacy, Alfonso R. Gennaro ed., Mack Publishing Co. Easton, Pa., 19th ed., 1995, Chapter 86 (hereby incorporated by reference). The formulation of gels and semisolids can be prepared, for example, by mixing the the VEGFR inhibitor (e.g., sunitinib) and any additional components or excipients in a standard V-blender.

### Kits

In a specific embodiment, the present invention is directed to kits comprising an Ang2 inhibitor (e.g., trebananib), a VEGFR inhibitor (e.g., sunitinib), and instructions for administration to patients, such as RCC patients. The kit instructions may indicate dosing amounts and regimens. In addition, the kit instructions will indicate that a patient's PLGF concentration (as discussed herein) may be compared to a PLGF reference concentration in accordance with the methods described herein. For example, the kit instructions may indicate that a patient's serum or plasma PLGF concentration should be determined and compared to a PLGF serum or plasma reference concentration in order to determine, e.g., whether a patient has an increased likelihood of obtaining clinical benefit from treatment with a therapeutically effective amount of a VEGFR inhibitor (e.g., sunitinib) and an Ang2 inhibitor (e.g., trebananib).

In another embodiment, the kits may comprise a compound (e.g., an antibody) capable of detecting and/or binding to PLGF. Such kits are useful in, e.g., the identification of patients who have an increased likelihood of obtaining clinical benefit from treatment with a therapeutically effective amount of a VEGFR inhibitor (e.g., sunitinib) and an Ang2 inhibitor (e.g., trebananib) by allowing for the determination of, e.g., a patient's PLGF concentration. The patient's PLGF concentration may then be compared to a PLGF reference concentration in accordance with the methods described herein.

In one embodiment, the instructions refer to a PLGF serum concentration of about 31.20 pg/mL or 31.20 pg/mL. In another embodiment, the instructions refer to a a PLGF plasma concentration of about 31.20 pg/mL or 31.20 pg/mL. In another embodiment, the instructions refer to a PLGF serum concentration of about 28.90 pg/mL or 28.90 pg/mL. In yet another embodiment, the instructions refer to a PLGF plasma concentration of about 28.90 pg/ml, or 28.90 pg/ml.

The above listings are by way of example only, and do not preclude the use of other compounds or treatments which can be used concurrently with the compounds described herein that are known by those skilled in the art or that could be arrived at by those skilled in the art using the guidelines set forth in this specification.

### EXAMPLES

The invention is further described with reference to the following non-limiting examples.

### Example 1

This Example describes a Phase 2, open label, multi-center study to estimate the efficacy and evaluate the safety and tolerability of trebananib in combination with sunitinib in the treatment of subjects with advanced clear cell carcinoma of the kidney. A more complete description of the study design is disclosed at clinicaltrials.gov, the disclosure of which is incorporated herein by reference.

The number of patients to be enrolled was approximately 80. Patients eligible for the study were at least 18 years of age. Both genders were eligible although no healthy patients were eligible. The primary objective was safety and tolerability, while the secondary outcomes included objective response rate, duration of response, PFS, OS and change in continuous measures of tumor burden.

| ARMS | ASSIGNED INTERVENTIONS |
|---|---|
| ARM A: | Drug: trebananib |
| Experimental Interventions: | 10 mg/kg IV (intravenous) weekly |
| Drug: trebananib | until unacceptable toxicity or disease |
| Drug: Sunitinib | progression |
| | Drug: Sunitinib |
| | 50 mg PO (orally) QD (once a day) |
| ARM B: | Drug: trebananib |
| Experimental Interventions: | 15 mg/kg IV weekly until unacceptable |
| Drug: trebananib | toxicity or disease progression |
| Drug: Sunitinib | Drug: Sunitinib |
| | 50 mg PO QD |

Inclusion Criteria were as follows:
1) Subjects must have a histologically confirmed metastatic RCC with a clear cell component.
2) Low or intermediate risk according to the Memorial Sloan Kettering Cancer Center (MSKCC) prognostic risk classification.
3) Measurable disease with at least one unidimensionally measurable lesion per RECIST guidelines with modifications.
4) Adequate organ and hematological function as evidenced by laboratory studies conducted at screening.
5) ECOG (Eastern Cooperative Oncology Group) performance status of 0 or 1.
6) LVEP≥45%

Exclusion Criteria were as follows:

### Disease Related

1) Primary tumor in situ. Patients must have had their primary tumor resected.
2) Known history of central nervous system metastasesSubjects who received radiation therapy must have recovered from all radiation induced toxicities prior to enrollment

### Medications

1) Currently or previously treated with sunitinib or other small molecule inhibitors of VEGF
2) Currently or previously treated with neutralizing antibodies of VEGF such as bevacizumab, or VEGF-TRAP
3) Currently or previously treated with trebananib, or other molecules that inhibit the angiopoietins or Tie2 receptor.

### General Medical

1) Known ongoing pancreatitis.
2) Myocardial infarction, cerebrovascular accident, transient ischemic attack, percutaneous transluminal coronary angioplasty/stent congestive heart failure, grade 2 or greater peripheral vascular disease, arrhythmias not controlled by outpatient medication, or unstable angina within 1 year prior to randomization.
3) Major surgery within 30 days before randomization or still recovering from prior surgery.
4) Uncontrolled hypertension as defined as diastolic>90 mmHg OR systolic>150 mmHg. Anti-hypertensive medications are permitted.

### Other

1) Other investigational procedures are excluded.
2) Subject currently is enrolled in or has not yet completed at least 30 days since ending other investigational device or drug study(s), or subject is receiving other investigational agent(s).

### Example 2

This example describes an analysis of the relationship between patient PLGF concentration and PFS (progression free survival) of patients enrolled in the phase 2 study described in Example 1.

Renal cancer patient's circulating levels of protein placental growth factor (PLGF) was analyzed along with a number of other analytes to determine if it was predictive of how well they will respond after treatment with trebananib and sunitinib. In a clinical trial (Example 1), patients with renal cancer were treated with 50 QD (once a day) sunitinib and either 10 mg/kg QW (once a week) trebananib or 15 mg/kg QW trebananib. Serum samples were collected prior to treatment (baseline) and used to measure circulating levels of the protein PLGF. The baseline patient PLGF concentration was analyzed to determine if it was informative of how well the patient would respond to the treatment regimens.

The patients PLGF concentrations were determined and tested for statistical association with progression free survival (PFS) time. Patient PLGF concentrations were found to be predictive of response to the combination therapy of trebananib and sunitinib (p=0.17), when patients were classified as to whether their baseline PLGF was “high” (above the median PLGF reference concentration) or “low” (below the median PLGF reference concentration). Patient classification was based on dichotomization by the overall median baseline PLGF (median PLGF reference concentration). Predictive significance was determined with a Cox proportional hazards model of PFS with the factors PLGF, cohort, and the interaction between PLGF and cohort. If the interaction factor had a p-value<0.05 then patient PLGF concentration was considered to be predictive of PFS.

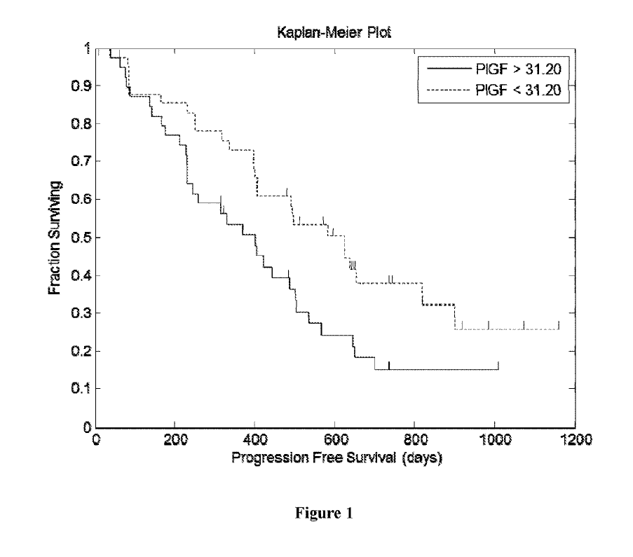

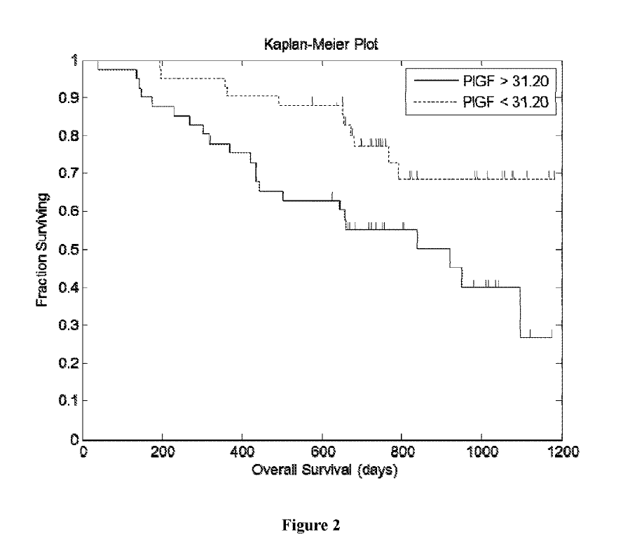

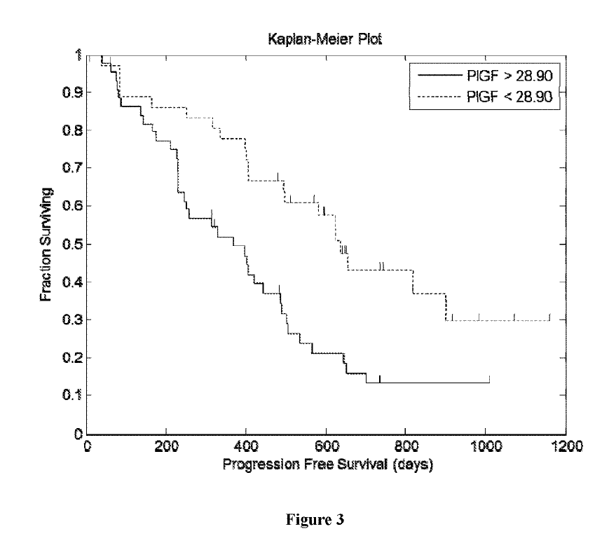

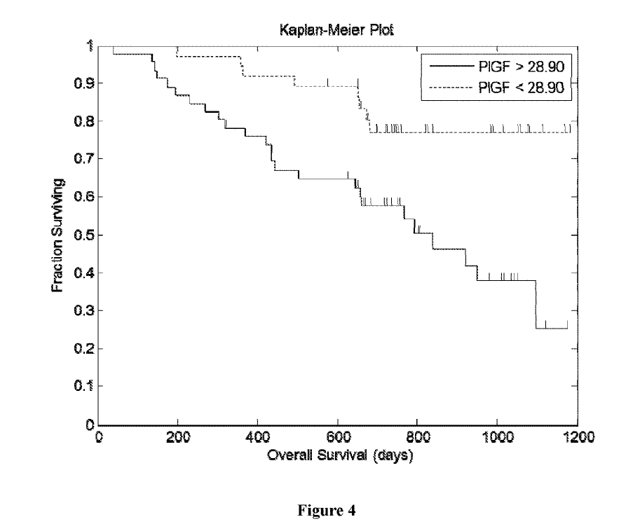

The relationship between PLGF and survival is illustrated graphically with Kaplan-Meier (KM) plots (FIGS. 1-4). The curves in the KM plots show how the fraction of patients having PFS (FIGS. 1 and 3) or OS (FIGS. 2 and 4) changes with time after the start of treatment. Differences between survival curves are evaluated with the Cox proportional hazards test. All patients were combined (i.e., 10 mg/kg and 1.5 mg/kg trebananib cohorts combined), and were classified into PLGF high and low groups. FIGS. 1 and 2 show the KM plots for PFS and OS respectively using a median PLGF cut-off of 31.2 pg/ml; FIGS. 3 and 4 use an optimal PLGF cut-off of 28.9 pg/ml. Those with low baseline patient PLGF concentration had longer PFS (FIG. 1, hazard ratio=2.23, p=0.026 and FIG. 2, hazard ratio=3.00, p=0.003). Similarly those with low baseline patient PLGF concentration had longer OS (FIG. 2, hazard ratio 2.52, p=0.012 and FIG. 4, hazard ratio 2.82. p=005).

The observation of a significant association between patient PLGF concentration and survival (PFS and OS) in trebananib+sunitinib treated patients indicates that baseline PLGF is surprisingly predictive of response to trebananib+sunitinib. Renal cancer patients with low patient PLGF concentrations are predicted to survive longer than those with high patient PLGF concentrations when they are treated with trebananib in combination with sunitinib.

1. A method of determining whether a human renal cell carcinoma (RCC) patient has an increased likelihood, or identifying a human RCC patient having an increased likelihood, of obtaining clinical benefit from treatment with a therapeutically effective amount of a vascular endothelial factor receptor (VEGFR) inhibitor and an angiopoietin 2 (Ang2) inhibitor, said method comprising
measuring the concentration of placental growth factor (PLGF) in an RCC patient sample; and
determining that the PLGF concentration in said RCC patient sample is lower than a PLGF reference concentration;
wherein a patient with a patient PLGF concentration lower than said PLGF reference concentration has an increased likelihood of obtaining clinical benefit from treatment with a therapeutically effective amount of a VEGFR inhibitor and an Ang2 inhibitor.

2. A method of treating a human renal cell carcinoma (RCC) patient with a therapeutically effective amount of a vascular endothelial factor receptor (VEGFR) inhibitor and an angiopoietin 2 (Ang2) inhibitor, said method comprising;
measuring the concentration of placental growth factor (PLGF) in an RCC patient sample;
determining that the PLGF concentration in said RCC patient sample is lower than a PLGF reference concentration; and
administering a therapeutically effective amount of a VEGFR inhibitor and an Ang2 inhibitor to said patient.

3. A method of treating a human renal cell carcinoma (RCC) patient having a patient placental growth factor (PLGF) concentration lower than a PLGF reference concentration, said method comprising:
administering a therapeutically effective amount of a vascular endothelial factor receptor (VEGFR) inhibitor and an angiopoietin 2 (Ang2) inhibitor to said patient.

4. The method of claim 2, wherein said human RCC patient is a patient having an increased likelihood of obtaining clinical benefit from treatment with a therapeutically effective amount of a vascular endothelial factor receptor (VEGFR) inhibitor and an angiopoietin 2 (Ang2) inhibitor.

5. (canceled)

6. The method according to claim 3, wherein said PLGF concentration in said RCC patient is a serum PLGF concentration.

7. The method according to claim 3, wherein said PLGF concentration in said RCC patient is a plasma PLGF concentration.

8. The method according to claim 3, further comprising obtaining an RCC sample from the patient.

9. The method according to claim 3, wherein said Ang2 inhibitor is selected from the group consisting of: trebananib, H4L4, CVX-060, MEDI3617, DX-2240, REGN910, CGI-1842, LC06, CGEN-25017, RG7594, CVX-241, LP-590, CEP-11981, MGCD265, regorafenib, and CrossMab

10. The method according to claim 3, wherein said Ang2 inhibitor is trebananib.

11. The method according to claim 3, wherein said VEGFR inhibitor is selected from the group consisting of: bevacizumab, pazopanib, sunitinib, axitinib, ponatinib, cabozantinib, lenvatinib, ramucirumab, regorafenib, vandetanib, and ziv-aflibercept.

12. The method according to claim 3, wherein said VEGFR inhibitor is sunitinib.

13-14. (canceled)

15. The method of claim 3, wherein said PLGF reference concentration is a value obtained from a statistical sampling of at least 50 RCC patients.

16. The method of claim 3, wherein said PLGF reference concentration is a PLGF plasma concentration from about 20 pg/mL to about 35 pg/mL.

17. The method of claim 3, wherein said PLGF reference concentration is a PLGF serum concentration from about 20 pg/mL to about 35 pg/mL.

18. The method of claim 3, wherein said VEGFR inhibitor is administered to the patient at a dose of about 50 mg.

19. The method of claim 3, wherein said Ang2 inhibitor is administered at doses of about 10 or about 15 mg/kg of patient body weight.

20. The method of claim 3, wherein said VEGFR inhibitor is administered at a dose of about 50 mg once daily (QD) on a 4-weeks-on/2-weeks-off schedule, and said Ang2 inhibitor is administered intravenously once a week (QW) at a dose of about 10 mg/kg or 15 mg/kg of patient body weight.
